(19) United States
(12) Patent Application Publication
(10) Pub. No.: US 2026/0334831 A1
(43) Pub. Date: Oct. 29, 2026

(54) IN-VEHICLE APPARATUS, FATIGUE LEVEL ESTIMATION SYSTEM, FATIGUE LEVEL ESTIMATION METHOD, AND NON-TRANSITORY STORAGE MEDIUM STORING COMPUTER PROGRAM

(71) Applicant: SUMITOMO ELECTRIC INDUSTRIES, LTD., Osaka-shi, Osaka (JP)

(72) Inventor: Junji YANO, Osaka-shi (JP)

(73) Assignee: SUMITOMO ELECTRIC INDUSTRIES, LTD., Osaka-shi, Osaka (JP)

(21) Appl. No.: 19/471,604

(22) PCT Filed: Mar. 1, 2024
(86) PCT No.: PCT/JP2024/007694

§ 371 Date: Oct. 1, 2025

(30) Foreign Application Priority Data
Apr. 3, 2023 (JP) 2023-059957

(51) Int. Cl. A61B 5/16; A61B 5/00; A61B 5/11; A61B 5/18

(52) U.S. Cl. CPC A61B 5/165; A61B 5/1118; A61B 5/18; A61B 5/6893; A61B 2503/22; A61B 2562/0247

(57) ABSTRACT

This in-vehicle device is mounted in a vehicle having a seat and a seat occupancy sensor provided to the seat, and comprises: a seat occupancy sensor output reception unit that receives sensor data from the seat occupancy sensor; a status information reception unit that receives status information representing the status of the vehicle; and a fatigue level estimation unit that is connected to the seat occupancy sensor output reception unit and the status information reception unit, and estimates the fatigue level of the driver of the vehicle who is seated in the seat provided with the seat occupancy sensor, on the basis of the sensor data and the status information.

### TECHNICAL FIELD

The present disclosure relates to an in-vehicle apparatus, a fatigue level estimation system, a fatigue level estimation method, and a computer program. This application is based upon and claims the benefit of priority from Japanese Patent Application No. 2023-059957, filed on Apr. 3, 2023, the disclosure of which is incorporated herein in its entirety by reference.

### BACKGROUND ART

Non-patent literature 1 discloses a method for estimating a fatigue level of a driver. Specifically, Non-patent literature 1 discloses a technique of estimating fatigue from the characteristics of a change in body pressure on the seat during long driving time by embedding strain gauge sensors at eight locations in both a seat back and a seat surface of the seat back of a vehicle and checking a change in body pressure during normal driving and during fatigue driving.

### CITATION LIST

### Non Patent Literature

Non-patent literature 1: Satoru Furugori, Yasuhiko Miura, Norimichi Yoshizawa, Chikara Iname, “ Estimation of Driver's Fatigue Based on Change in Body Pressure Distribution ”, Mazda Technical Research Center, Apr. 19, 2004, p. 161 to 166

### SUMMARY OF INVENTION

An in-vehicle apparatus according to a first aspect of the present disclosure is an in-vehicle apparatus to be mounted on a vehicle including a seat and a seating sensor provided on the seat. The in-vehicle apparatus includes a seating sensor output receiving unit configured to receive sensor data from the seating sensor, a state information receiving unit configured to receive state information indicating a state of the vehicle, and a fatigue level estimation unit connected to the seating sensor output receiving unit and the state information receiving unit and configured to estimate a fatigue level of a driver of the vehicle seated on the seat provided with the seating sensor, based on the sensor data and the state information.

### BRIEF DESCRIPTION OF THE DRAWINGS

FIG. 1 is a functional block diagram of an in-vehicle system according to a first embodiment of the present disclosure.

FIG. 2 is a schematic diagram showing an example of the center of gravity of the body pressure detected by the seating sensor.

FIG. 3 is a flowchart showing a control structure of a computer program for implementing an in-vehicle apparatus according to a first embodiment of the present disclosure.

FIG. 4 is a schematic diagram for explaining a method of detecting a fatigue level by an in-vehicle apparatus according to a first embodiment of the present disclosure.

FIG. 5 is a schematic diagram for explaining a method of detecting a fatigue level of an in-vehicle apparatus according to a modification of the first embodiment of the present disclosure.

FIG. 6 is a functional block diagram of an in-vehicle system according to a second embodiment of the present disclosure.

FIG. 7 is a schematic diagram showing selection of a map for mental fatigue in an in-vehicle system according to a second embodiment.

FIG. 8 is a schematic diagram showing selection of a map for eye fatigue in an in-vehicle system according to a second embodiment.

FIG. 9 is a flowchart showing a control structure of a program for implementing an in-vehicle system according to a second embodiment of the present disclosure.

FIG. 10 is a flowchart showing the control structure of a program that performs a process of calculating a mental fatigue accumulation probability.

FIG. 11 is a flowchart showing a control structure of a program that performs a process for calculating an eye fatigue accumulation probability.

FIG. 12 is a schematic diagram showing a schematic configuration of a fatigue management system according to a third embodiment of the present disclosure.

FIG. 13 is a functional block diagram of an in-vehicle system of a fatigue management system according to a third embodiment of the present disclosure.

FIG. 14 is a functional block diagram of a fatigue management server of a fatigue management system according to a third embodiment of the present disclosure.

FIG. 15 is a hardware block diagram of a computer that executes an in-vehicle apparatus according to each embodiment of the present disclosure.

FIG. 16 is a hardware block diagram of a fatigue management server according to a third embodiment of the present disclosure.

### DETAILED DESCRIPTION

A main cause of a traffic accident is inattentive driving by a fatigue. Inattentive driving by a fatigue represents about one fourth of traffic accidents. In order to avoid such traffic accidents, it is essential to accurately detect the fatigue state of the driver, ensure that they take appropriate rest and vacation, and alleviate the workload.

However, the fatigue may be different from the sense of the person himself/herself. That is, even when the person does not feel fatigued, their body may be actually fatigued. Thus, it is necessary to have an external objective standard for determining whether the driver is fatigued, rather than leaving the judgment to the driver themselves.

In order to solve such a problem, it is also possible to attach a plurality of sensors to the driver or attach a so-called smart watch to the driver. However, it is not practical to mount a plurality of sensors on many drivers. Also, smart watches are often personal items and not worn by many drivers.

### Problems to be Solved by Present Disclosure

According to the technique disclosed in the above Non-patent literature 1, an effect is obtained that the fatigue of the driver can be estimated without placing a burden on the driver. However, there is still room for improvement in order to put this technology into practical use. This is a problem that the estimation accuracy is assumed to decrease due to the driver performing an unexpected motion during actual traveling.

Thus, an object of the present disclosure is to provide an in-vehicle apparatus, a fatigue level estimation system, a fatigue level estimation method, and a computer program that can estimate a fatigue level of a driver more accurately and perform appropriate process without placing a burden on the driver.

### Advantageous Effects of Present Disclosure

As described above, according to the present disclosure, it is possible to provide an in-vehicle apparatus, a fatigue level estimation system, a fatigue level estimation method, and a computer program that can estimate a fatigue level of a driver more accurately and perform appropriate process without placing a burden on the driver.

### Description of Embodiments of Present Disclosure

In the following description and the drawings, the same components are denoted by the same reference numerals. Thus, detailed description thereof will not be repeated. It is noted that, at least some of the embodiments described below may be combined as desired.
(1) An in-vehicle apparatus according to a first aspect of the present disclosure is an in-vehicle apparatus to be mounted on a vehicle including a seat and a seating sensor provided on the seat. The in-vehicle apparatus includes a seating sensor output receiving unit configured to receive sensor data from the seating sensor, a state information receiving unit configured to receive state information indicating a state of the vehicle, and a fatigue level estimation unit connected to the seating sensor output receiving unit and the state information receiving unit and configured to estimate a fatigue level of a driver of the vehicle seated on the seat provided with the seating sensor, based on the sensor data and the state information. With this configuration, the fatigue level of the driver can be estimated more accurately and an appropriate process can be performed without placing a burden on the driver.
(2) In the above (1), the fatigue level estimation unit may include a fatigue level increasing unit configured to increase a fatigue level parameter based on the sensor data when the state information indicates that the vehicle is traveling, and a fatigue level decreasing unit configured to decrease the fatigue level parameter when the state information indicates that the vehicle is not traveling. With this configuration, without placing a burden on the driver, by considering both the time when the vehicle is traveling and the time when the vehicle is stopped, the fatigue level of the driver can be estimated more accurately and an appropriate process can be performed.
(3) In the above (2), the fatigue level increasing unit may include a deviation calculation unit configured to repeatedly calculate a deviation determined between the sensor data and reference data over a period of time during the state information indicating that the vehicle is traveling, the reference data being acquired in advance from a sitting posture leading to less fatigue, a first determination unit configured to determine whether the deviation is greater than or equal to a threshold value in response to the deviation being calculated by the deviation calculation unit, and an addition unit configured to add a first value to the fatigue level in response to a determination by the first determination unit that the deviation is greater than or equal to the threshold value. With this configuration, the appropriate process can be performed by estimating the fatigue level of the driver more accurately without placing a burden on the driver based on both the vehicle's traveling condition and the sensor data from the seating sensor.
(4) In the above (3), the reference data may include center-of-gravity position information indicating a first center-of-gravity position of a seated person in the sitting posture leading to less fatigue, the first center-of-gravity position being based on body pressure exerted on the seating sensor by the seated person. The deviation calculation unit may include a deviation storage unit configured to store the deviation, a center-of-gravity position calculation unit configured to calculate a second center-of-gravity position of the driver based on body pressure exerted on the seating sensor by the driver, based on the sensor data over the period of time during the state information indicating that the vehicle is traveling, a distance calculation unit configured to calculate a distance defined between the first center-of-gravity position and the second center-of-gravity position, and a distance addition unit configured to add the distance calculated by the distance calculation unit to the deviation stored in the deviation storage unit. With this configuration, based on the vehicle's traveling condition, the sensor data from the seating sensor, and the reference data, the fatigue level of the driver can be estimated accurately according to a more objective standard without placing a burden on the driver, allowing for appropriate processes to be taken.
(5) In the above (3), the reference data may include a reference distribution, the reference distribution being a distribution of body pressure exerted on the seating sensor by a seated person in the sitting posture leading to less fatigue. The deviation calculation unit may include a deviation storage unit configured to store the deviation, a body pressure distribution measuring unit configured to periodically measure a body pressure distribution of the driver, based on the sensor data over the period of time during the state information indicating that the vehicle is traveling, the body pressure distribution being a distribution of body pressure exerted on the seating sensor by the driver, a distance calculation unit configured to calculate a distance defined between the body pressure distribution and the reference distribution, and a distance addition unit configured to add the distance calculated by the distance calculation unit to the deviation stored in the deviation storage unit. With this configuration, based on the vehicle's traveling condition, the sensor data from the seating sensor, and the reference data, the fatigue level of the driver can be estimated accurately according to a more objective standard without placing a burden on the driver, allowing for appropriate processes to be taken.
(6) In any one of the above (2) to (5), the fatigue level increasing unit may include a fatigue level storage unit configured to store a value of the fatigue level, and a movement amount calculation unit configured to calculate an amount of movement of a center-of-gravity position of the driver based on body pressure exerted on the seating sensor by the driver, based on the sensor data over a period of time during the state information indicating that the vehicle is traveling, and add the amount of movement to the value of the fatigue level. With this configuration, the fatigue level of the driver can be accurately estimated according to a more objective standard and an appropriate process can be performed without placing a burden on the driver based on the vehicle's traveling condition and the movement of the driver during driving, which is assumed from the sensor data from the seating sensor.
(7) In any one of the above (3) to (6), the fatigue level estimation unit may further include a first probability calculation unit configured to calculate a probability of mental fatigue accumulation in the driver, based on a traveling environment of the vehicle, a second determination unit configured to determine whether the probability calculated by the first probability calculation unit is greater than or equal to a threshold value, and a mental fatigue level addition unit configured to add a second value to the fatigue level in response to a determination by the second determination unit that the probability is greater than or equal to the threshold value. With this configuration, the fatigue level of the driver is accurately estimated according to a more objective standard without placing a burden on the driver on the basis of the vehicle's traveling condition, the sensor data from the seating sensor, and the probability of mental fatigue accumulation, and an appropriate process can be performed.
(8) In the above (7), the fatigue level estimation unit may further include a second probability calculation unit configured to calculate a probability of eye fatigue accumulation in the driver, based on the traveling environment of the vehicle, a third determination unit configured to determine whether the probability calculated by the second probability calculation unit is greater than or equal to a threshold value, and an eye fatigue level addition unit configured to add a third value to the fatigue level in response to a determination by the third determination unit that the probability is greater than or equal to the threshold value. With this configuration, the fatigue level of the driver is accurately estimated according to a more objective standard without placing a burden on the driver based on the vehicle's traveling condition, the sensor data from the seating sensor, the probability of mental fatigue accumulation, and the probability of eye fatigue accumulation, and an appropriate process can be performed.
(9) In any one of the above (3) to (6), the fatigue level estimation unit may further include a first probability calculation unit configured to calculate a probability of eye fatigue accumulation in the driver, based on a traveling environment of the vehicle, a second determination unit configured to determine whether the probability calculated by the first probability calculation unit is greater than or equal to a threshold value, and an eye fatigue level addition unit configured to add a second value to the fatigue level in response to a determination by the second determination unit that the probability is greater than or equal to the threshold value. With this configuration, the fatigue level of the driver is accurately estimated according to a more objective standard without placing a burden on the driver on the basis of the vehicle's traveling condition, the sensor data from the seating sensor, and the probability of eye fatigue accumulation, and an appropriate process can be performed.
(10) A fatigue level estimation system according to a second aspect of the present disclosure is a fatigue level estimation system for estimating a fatigue level of a driver of a vehicle including a seating sensor, the fatigue level estimation system including an in-vehicle apparatus to be mounted on the vehicle, and a server configured to communicate with the in-vehicle apparatus. The in-vehicle apparatus includes a seating sensor output receiving unit configured to receive sensor data from the seating sensor, a state information receiving unit configured to receive state information indicating a state of the in-vehicle with the in-vehicle apparatus, and a transmitting unit configured to transmit the sensor data received by the seating sensor output receiving unit and the state information received by the state information receiving unit to the server. The server includes a fatigue level estimation unit configured to receive the sensor data and the state information from the in-vehicle apparatus and estimate a fatigue level of the driver of the vehicle seated on a seat provided with the seating sensor, based on the sensor data and the state information, and a process execution unit configured to execute a predetermined process for reducing fatigue of the driver in response to the fatigue level estimated by the fatigue level estimation unit exceeding a threshold value. With this configuration, the fatigue level of the driver can be estimated more accurately and an appropriate process can be performed without placing a burden on the driver.
(11) A fatigue level estimation method according to a second aspect of the present disclosure is a fatigue level estimation method for estimating a fatigue level of a driver seated on a seat of a vehicle based on sensor data from a seating sensor provided on the seat. The fatigue level estimation method includes receiving the sensor data from the seating sensor, receiving state information indicating a state of the vehicle, and estimating a fatigue level of the driver based on the sensor data and the state information. With this configuration, the fatigue level of the driver can be estimated more accurately and an appropriate process can be performed without placing a burden on the driver.
(12) A computer program according to a fourth aspect of the present disclosure is a computer program for causing a computer to estimate a fatigue level of a driver seated on a seat of a vehicle based on sensor data from a seating sensor provided on the seat, the computer program causing the computer to execute receiving the sensor data from the seating sensor, receiving state information indicating a state of the vehicle, and estimating a fatigue level of the driver based on the sensor data and the state information. With this configuration, the fatigue level of the driver can be estimated more accurately and an appropriate process can be performed without placing a burden on the driver.

The present disclosure can not only be embodied as an in-vehicle apparatus equipped with such a distinctive processing unit but also be implemented as a fatigue management method that incorporates the corresponding distinctive processes, or as a program for executing those step by a computer. The present disclosure can also be implemented as a semiconductor integrated circuitry that implements a part or all of the in-vehicle apparatus, or as a fatigue management system including the in-vehicle apparatus.

### DETAILS OF EMBODIMENTS OF PRESENT DISCLOSURE

Specific examples of an in-vehicle apparatus, a fatigue level estimation system, a fatigue level estimation method, and a computer program according to embodiments of the present disclosure will be described below with reference to the drawings. It is noted that, the present disclosure is not limited to these examples, but is defined by the scope of the claims, and is intended to include all modifications within the meaning and scope equivalent to the scope of the claims.

### 1. First Embodiment

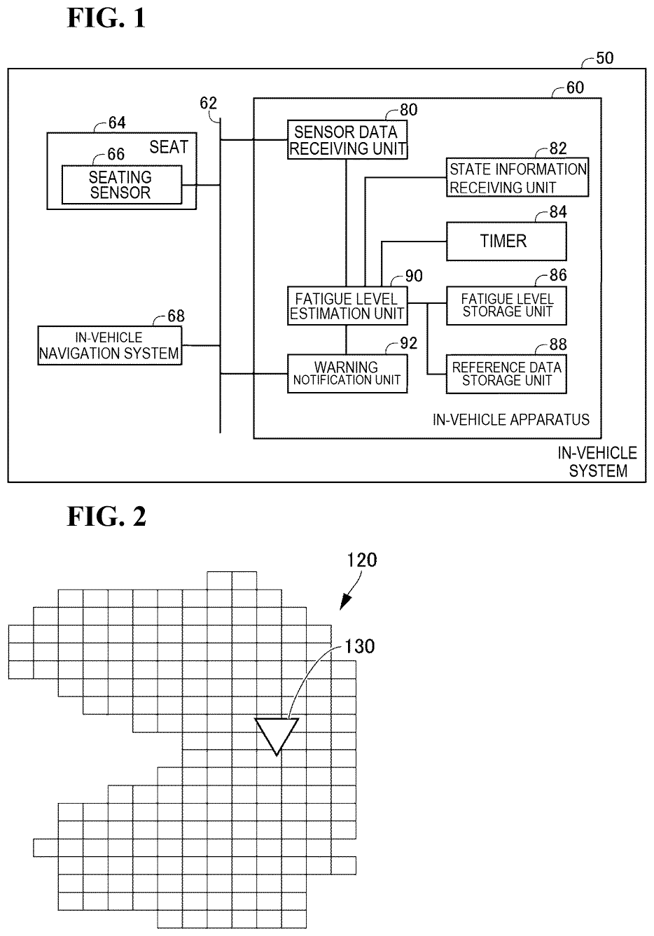

FIG. 1 shows a functional block diagram of an in-vehicle system 50 according to a first embodiment of the present disclosure. Referring to FIG. 1, the in-vehicle system 50 includes a seating sensor 66, an in-vehicle network 62, an in-vehicle navigation system 68, and an in-vehicle apparatus 60. The seating sensor 66 is provided in a seat 64 for the driver in the vehicle, and periodically measures the distribution of the body pressure of the driver seated on the seat 64, and outputs the distribution as sensor data. It is noted that, the seating sensor 66 may randomly measure the distribution of the body pressure of the driver. The seating sensor 66 is connected to the in-vehicle network 62. The in-vehicle navigation system 68 is connected to the in-vehicle network 62. The in-vehicle apparatus 60 is connected to the in-vehicle network 62. The in-vehicle apparatus 60 further estimates a fatigue level of the driver, and executes a predetermined process such as prompting the driver to take a rest when the estimated fatigue level exceeds a certain threshold value. The in-vehicle network 62 may be any of the following: a controller area network (CAN), a local interconnect network (LIN), a media oriented systems transport (MOST), FlexRay, 10BASE-T1S, 100BASE-T1, 1000BASE-T1, a clock extension peripheral interface (CXPI), and an automotive safety restraints bus (ASRB) CAN XL, etc. In this embodiment, a CAN is used as the in-vehicle network 62, although not particularly limited thereto.

The in-vehicle apparatus 60 includes a sensor data receiving unit 80, a state information receiving unit 82, a timer 84, and a reference data storage unit 88. The sensor data receiving unit 80 is connected to the in-vehicle network 62 and receives sensor data from the seating sensor 66 via the in-vehicle network 62. The state information receiving unit 82 receives state information indicating the state (traveling or non-traveling (that is, stopped or parked)) of the in-vehicle with the in-vehicle system 50 from a vehicle control unit (not shown). The reference data storage unit 88 stores reference data that is referred to for determining the fatigue state of the driver.

The in-vehicle apparatus 60 further includes a fatigue level estimation unit 90 (including a calculation unit, an addition unit, a measuring unit, and a determination unit and the same applies hereinafter.), a fatigue level storage unit 86, and a warning notification unit 92. A fatigue level estimation unit 90 receives the sensor data from the sensor data receiving unit 80. The fatigue level estimation unit 90 further executes a process of periodically estimating the fatigue level of the driver under the control of the timer 84 based on the state information received by the state information receiving unit 82 and the reference data stored in the reference data storage unit 88. It is noted that, the fatigue level estimation unit 90 may execute a process of estimating the fatigue level of the driver at random. A fatigue level storage unit 86 stores the fatigue level estimated by the fatigue level estimation unit 90. The warning notification unit 92 notifies the driver of a warning to take a rest using the in-vehicle navigation system 68 in response to the fact that the fatigue level estimated by the fatigue level estimation unit 90 becomes greater than or equal to a predetermined threshold value.

FIG. 2 shows an outline of a reference data 120 stored in the reference data storage unit 88 shown in FIG. 1. Referring to FIG. 2, the reference data 120 includes, for example, a sitting posture. The reference data 120 is a center-of-gravity position of the body pressure obtained from a set of body pressure values of each block indicated by a rectangular grid in FIG. 2. Each block indicates each of a plurality of rectangular regions when dividing the area of the seat 64 into them. That is, the respective blocks indicate a body pressure distribution. The reference data 120 is obtained from the output of the seating sensor 66 when the person in a state of not fatigued is seated on the seat 64, for example. The reference data 120 can derive the position of a center of gravity 130 of the body pressure by using the position of each block and the body pressure value of each block. The center of gravity 130 is the reference data 120 in this embodiment. The reference data 120 may be calculated from the output of the seating sensor 66 when a person other than the driver is seated on the seat 64. It is noted that, in this embodiment, not only the value of the center of gravity 130 but also the body pressure value in each region is included in the reference data 120.

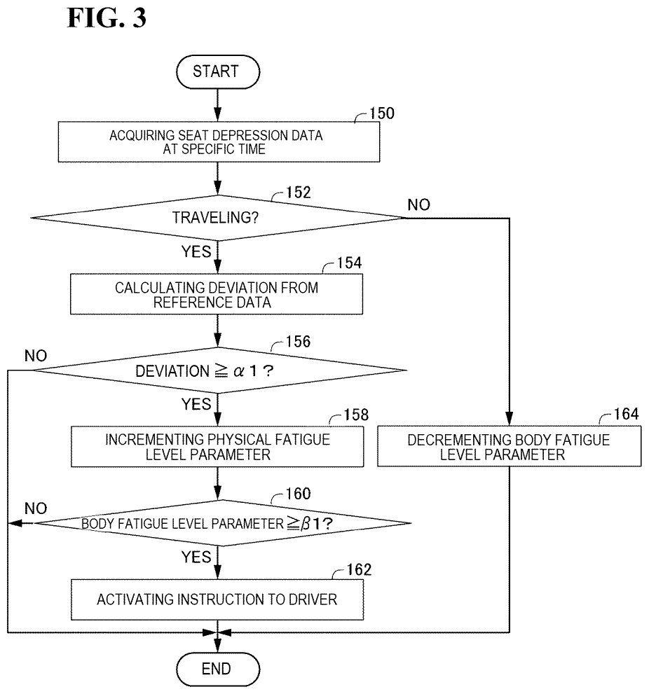

FIG. 3 shows a control structure of a program executed by the fatigue level estimation unit 90 to estimate the fatigue level of the driver. In the following description, the “physical fatigue level parameter” is a specific value indicating the physical fatigue level of the driver. This value is reset to zero, for example, when the driver first sits on the seating sensor 66 each day. Alternatively, this value may be reset when, for example, the administrator determines that the driver's condition is good.

Referring to FIG. 3, this program includes step 150 of acquiring sensor data indicating seat depression data (body pressure data) at a specific time based on a constant cycle, and step 152 of branching the flow of control according to whether the in-vehicle with the in-vehicle system 50 is in a traveling condition based on the state information from the state information receiving unit 82.

The program further includes step 154 of calculating a deviation between reference data and the seat depression data acquired in step 150 when the determination in step 152 is YES.

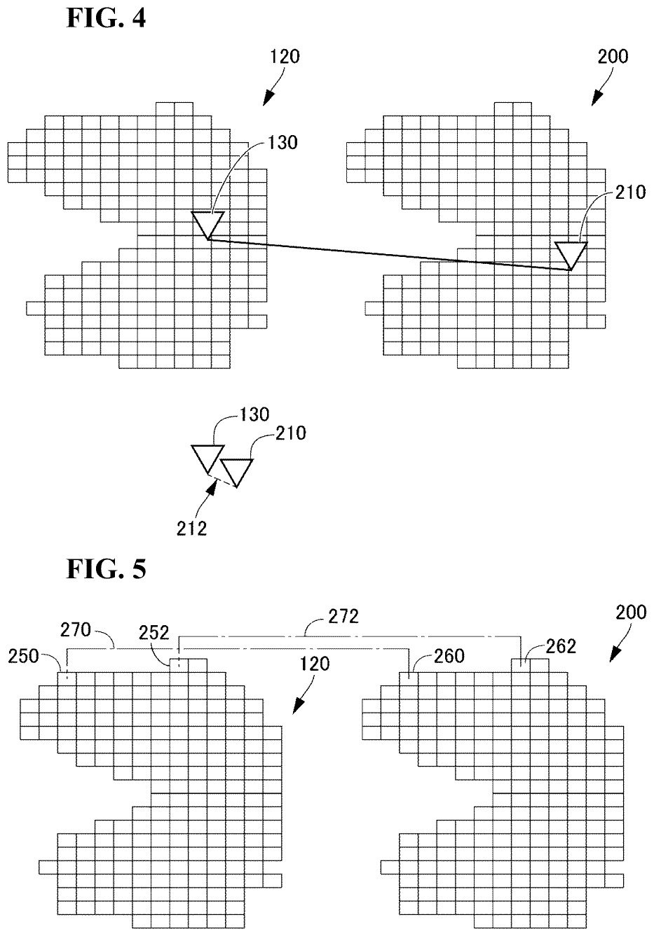

A method of calculating the deviation according to the first embodiment will be described with reference to FIG. 4. In this embodiment, by comparing the position of the center of gravity 130 of the reference data 120 and the position of a center of gravity 210 of a seat depression data 200 acquired in step 150, the deviation would be obtained from the distance 212 between them. It is noted that, in this embodiment, the Euclidean distance is used as the distance, but any index may be used as long as it can be used as the distance.

Referring again to FIG. 3, the program further includes step 156 for branching the flow of control according to whether the deviation calculated in step 154 is greater than or equal to a predetermined threshold value α1, and step 158 for incrementing the physical fatigue level parameter (adding the first value as addition unit) when the determination in step 156 is YES.

The program further includes step 160 for branching the flow of control depending on whether the physical fatigue level parameter after being incremented in step 160 is greater than or equal to a threshold value β1, and step 162 for activating an instruction to prompt the driver to take a rest and ending the execution of the program when the determination in step 160 is YES.

When the determination in step 156 is NO and when the determination in step 160 is NO, the execution of the program ends immediately.

The program further includes step 164, which decrements the physical fatigue level parameter when the determination in step 152 is NO and ends the execution of the program.

The physical fatigue level parameters after being incremented in step 158 and the physical fatigue level parameter after being decremented in step 164 are stored in the fatigue level storage unit 86 shown in FIG. 1, and are read out when either step 158 or step 164 is executed next. It is noted that, the value to be incremented and the value to be decremented may be equal to each other or may be different from each other. These values, the threshold value α1, and a threshold value β are design specifications that are determined by a designer through, for example, an experiment or the like at the time of design, depending on the execution interval of the program. It is considered normal that the value decremented in step 164 is greater than the value incremented in step 158.

For example, the program determines the increment value in step 158 so that the determination in step 160 of FIG. 3 becomes YES when the duration for which the deviation exceeds the threshold value continuously exceeds one hour. Thereafter, the program may determine the decrement value to the extent that the fatigue level is subtracted to substantially zero when the period during the driver resting, that is, the period during the determination in step 152 NO, elapses by about 15 minutes. It is noted that, in this embodiment, the period when the vehicle is not traveling includes, for example, the period spent waiting at traffic signals. However, this embodiment is not limited to such an embodiment, and the determination in step 152 may be made NO only when the engine is stopped (or the drive motor is stopped).

Further, the program does not need to activate the instruction even when the determination in step 160 is continuously YES for a predetermined time after the activation of the instruction in step 162.

According to this embodiment, the program acquires seat depression data of the driver at a predetermined cycle after the driver starts driving (step 150). When the vehicle is not traveling, the program calculates the center-of-gravity position of the body pressure based on the seat depression data, and calculates the distance from the center-of-gravity position of the reference data (step 154). This program increments the physical fatigue level parameter in step 160 when the deviation is equal to or larger than the threshold value al, with the distance as the deviation. When the physical fatigue level parameter becomes equal to or greater than the threshold value β1, the program activates an instruction to prompt the driver to take a rest.

During the time that the vehicle is stopped while traveling, and while the driver is taking a rest, the determination in step 152 is NO and the physical fatigue level parameter is decremented in step 164. As a result, it is possible to give an instruction to take a rest to the driver by an objective index. When sufficient rest is not taken, the physical fatigue level parameter will not completely reach zero, and the time until the next rest instruction will become shorter. Thus, as long as the driver follows the instruction of the in-vehicle apparatus 60, the driver can drive the vehicle with a sufficient rest.

### Modification of First Embodiment

In the first embodiment, as shown in FIG. 4, the distance 212 between the center of gravity 130 of the reference data 120 and the center of gravity 210 of seat depression data 200 obtained as a sensor output is calculated and used as an index indicating whether to increment the body fatigue level parameter. However, the disclosure is not limited to such embodiments.

FIG. 5 shows a method of calculating a deviation in the modification of the first embodiment. Referring to FIG. 5, in this modification, the program calculates the difference between the body pressure value of each region including the reference data 120 and the body pressure value of each region including the seat depression data 200 between the corresponding regions, and sums the differences over the entire region. The obtained value is a deviation.

For example, the program calculates a difference 270 in body pressure value between a region 250 of the reference data 120 and a region 260 corresponding to the region 250 in the seat depression data 200. Similarly, the program calculates a difference 272 in body pressure value between another region, for example, a region 252 of the reference data 120 and a region 262 corresponding to the region 252 in the seat depression data 200. After calculating the differences between each region, the sum is calculated to determine the deviation.

It is noted that, as the difference in body pressure value between the two regions, the absolute value of the difference between the two regions may be used, or the square of the difference between the two regions may be used.

Even when the deviation is calculated in this manner, the same effect as that of the first embodiment can be obtained. It is noted that, it is expected that the value of deviation in this modification is larger than the value of deviation in the first embodiment. However, by adjusting the value of the threshold value al shown in FIG. 3, the in-vehicle system 50 can perform similarly and achieve comparable effects despite the difference in the value of the deviation.

As the deviation, not only the above-described ones but also other various indices can be used. The program may use, as the deviation, a difference between a sum of the amount of movement of the center-of-gravity position of the body pressure of the driver during driving per unit time and a normal amount of movement. Further, the program may use not only the position of the center of gravity but also the moving speed of the center of gravity, the acceleration, and the like. Furthermore, for example, a statistical value such as an average value or a variance of the amount of movement of the center of gravity per unit time can be used for the calculation of the deviation.

### 2. Second Embodiment

In the first embodiment, only the driver's body fatigue is taken into consideration. However, the disclosure is not limited to such embodiments. Fatigue to be considered may include, for example, mental fatigue and eye fatigue. It is considered that it is desirable to consider these factors in order to drive the vehicle safely. The second embodiment relates to such an embodiment. It is noted that, in the second embodiment, the comparison of the position of the center of gravity as described in the first embodiment is used as the reference data. However, this embodiment is not limited to such a form, and reference data according to the modification described in the first embodiment may be used, or the amount of movement of the center of gravity may be used.

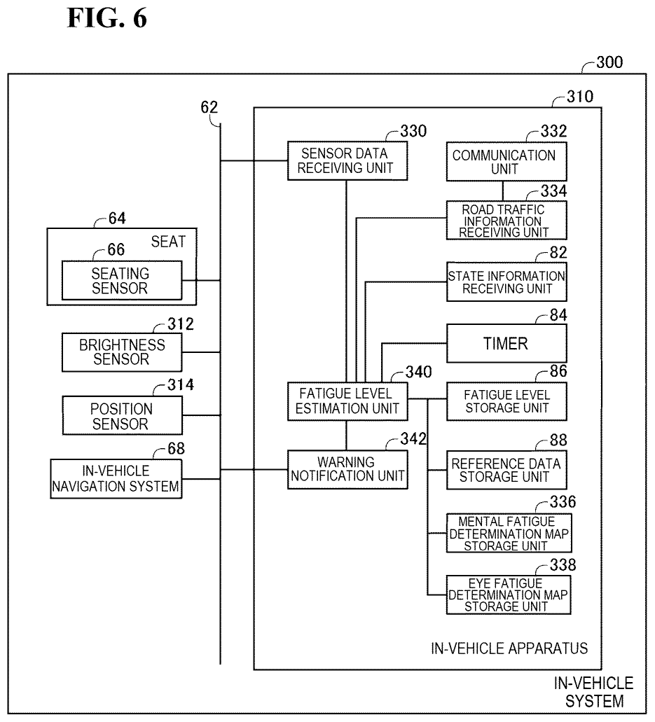

FIG. 6 shows a functional block diagram of an in-vehicle system 300 according to the second embodiment of the present disclosure. Referring to FIG. 6, the in-vehicle system 300 includes the in-vehicle network 62, the seat 64, the seating sensor 66, and the in-vehicle navigation system 68 similar to those shown in FIG. 1.

The in-vehicle system 300 further includes a brightness sensor 312 and a position sensor 314. The brightness sensor 312 is connected to the in-vehicle network 62, measures the ambient brightness around the in-vehicle with the in-vehicle system 300, and outputs sensor data representing the brightness. The position sensor 314 is connected to the in-vehicle network 62, detects the position of the vehicle, and outputs position information.

The in-vehicle system 300 further includes, in place of the in-vehicle apparatus 60 shown in FIG. 1, an in-vehicle apparatus 310 that performs a process of calculating a fatigue level of the driver using sensor data from the seating sensor 66, sensor data from the brightness sensor 312, and sensor data from the position sensor 314, and notifying the driver of a warning instructing the driver to take a rest using the in-vehicle navigation system 68 when the fatigue level becomes greater than or equal to a predetermined threshold value.

The in-vehicle apparatus 310 includes a sensor data receiving unit 330, a communication unit 332, a road traffic information receiving unit 334, and the state information receiving unit 82, the timer 84, the fatigue level storage unit 86 and the reference data storage unit 88 similar to those shown in FIG. 1. The sensor data receiving unit 330 is connected to the in-vehicle network 62 and receives sensor data from the seating sensor 66, the brightness sensor 312, and the position sensor 314. The road traffic information receiving unit 334 receives road traffic information from a road traffic information server (not shown) that provides road traffic information via the communication unit 332.

The in-vehicle apparatus 310 further includes a mental fatigue determination map storage unit 336, an eye fatigue determination map storage unit 338, a fatigue level estimation unit 340, and a warning notification unit 342. The mental fatigue determination map storage unit 336 stores a map for mental fatigue determination, which is used by the in-vehicle apparatus 310 to estimate mental fatigue. The eye fatigue determination map storage unit 338 stores a map for eye fatigue determination, which is used by the in-vehicle apparatus 310 to estimate eye fatigue. The fatigue level estimation unit 340 executes a process of periodically estimating the fatigue level of the driver under the control of the timer 84 based on the sensor data from the sensor data receiving unit 330, the road traffic information from the road traffic information receiving unit 334, the state information of the vehicle from the state information receiving unit 82, the reference data stored in the reference data storage unit 88, the map for mental fatigue determination stored in the mental fatigue determination map storage unit 336, and the map for eye fatigue determination stored in the eye fatigue determination map storage unit 338. It is noted that, the fatigue level estimation unit 340 may execute a process of estimating the fatigue level of the driver at random. The warning notification unit 342 notifies the driver of a warning to take a rest using the in-vehicle navigation system 68 in response to the fact that the fatigue level estimated by the fatigue level estimation unit 340 becomes greater than or equal to a predetermined threshold value.

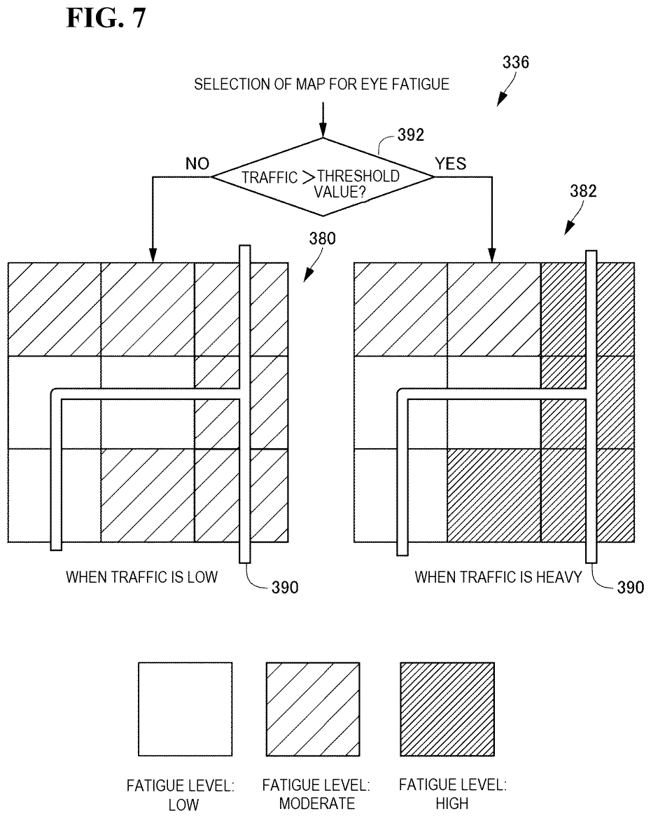

FIG. 7 shows the configuration of the map for mental fatigue determination stored in the mental fatigue determination map storage unit 336. Referring to FIG. 7, the mental fatigue determination map storage unit 336 includes a first mental fatigue determination map 380 used when the traffic is equal to or less than a threshold value and a second mental fatigue determination map 382 used when the traffic is greater than the threshold value. It is noted that, traffic is an example of a traveling environment.

The first mental fatigue determination map 380 and the second mental fatigue determination map 382 are maps related to the same region. For example, the first mental fatigue determination map 380 divides each area into a plurality of regions and stores the relationships between each region and roads 390 within the regions. Further, each of the plurality of regions is assigned a value that indicates the probabilities of mental fatigue accumulation for the driver when a vehicle is traveling within that region under conditions of low traffic volume. In the example shown in FIG. 7, a region where the probability of mental fatigue accumulation is small is indicated by white, and a region where the probability of mental fatigue accumulation is medium is indicated by coarse hatching. Further, in the second mental fatigue determination map 382, a region having a high probability of mental fatigue accumulation is indicated by denser hatching. The probability of mental fatigue accumulation is small as a whole in the first mental fatigue determination map 380, and conversely, is large in the second mental fatigue determination map 382.

The fatigue level estimation unit 340 specifies a region in which the in-vehicle with the in-vehicle system 300 is currently traveling. The fatigue level estimation unit 340 further determines, based on the road traffic information obtained from the road traffic information receiving unit 334, whether the traffic volume in the region where the vehicle is currently traveling exceeds a certain threshold value (determination 392) and selects the second mental fatigue determination map 382 when the traffic volume is greater than the threshold value, or selects the first mental fatigue determination map 380 otherwise. The fatigue level estimation unit 340 further identifies a region in which the vehicle is traveling in the selected map, and determines the accumulation probability of the mental fatigue assigned to the region. This value is used when the fatigue level estimation unit 340 estimates the fatigue level of the driver.

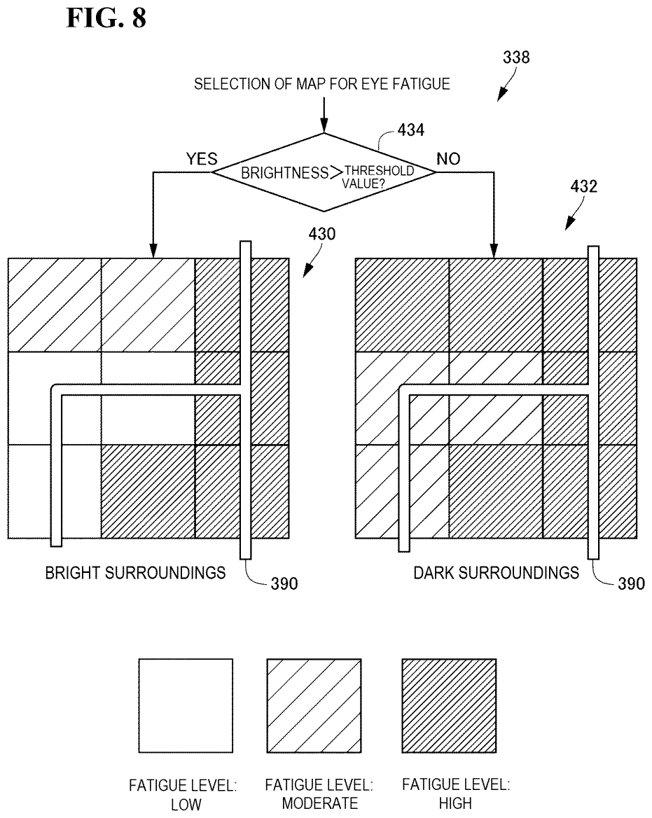

FIG. 8 shows the configuration of the eye fatigue determination map stored in the eye fatigue determination map storage unit 338. Referring to FIG. 8, the eye fatigue determination map storage unit 338 includes a first eye fatigue determination map 430 used when the ambient brightness is greater than the threshold value, and a second eye fatigue determination map 432 used when the ambient brightness is equal to or less than the threshold value.

The first eye fatigue determination map 430 and the second eye fatigue determination map 432 are maps related to the same region as the first mental fatigue determination map 380 shown in FIG. 7. For example, the first eye fatigue determination map 430 divides each area into a plurality of regions and stores the relationships between each region and the roads 390 within the regions. Further, each of the plurality of regions is assigned a value that indicates the probabilities of eye fatigue accumulation for the driver when a vehicle is traveling within that region under conditions of low traffic volume. In the example shown in FIG. 8, a region where the probability of eye fatigue accumulation is small is indicated by white, and a region where the probability of eye fatigue accumulation is medium is indicated by coarse hatching. Further, in the second eye fatigue determination map 432, a region having a high probability of eye fatigue accumulation is indicated by denser hatching. Then, the probability of eye fatigue accumulation is small as a whole in the first eye fatigue determination map 430, and conversely, is large in the second eye fatigue determination map 432.

The fatigue level estimation unit 340 specifies a region in which the in-vehicle with the in-vehicle system 300 is currently traveling. The fatigue level estimation unit 340 further determines, based on the sensor data indicating brightness from the brightness sensor 312 (see FIG. 6), whether the ambient brightness around the vehicle currently exceeds a threshold value (determination 434), and selects the first eye fatigue determination map 430 when the traffic volume exceeds the threshold value, or selects the second eye fatigue determination map 432 otherwise. The fatigue level estimation unit 340 further identifies a region in which the vehicle is traveling in the selected map, and determines the accumulated probability of eye fatigue assigned to the region. This value is used when the fatigue level estimation unit 340 estimates the fatigue level of the driver.

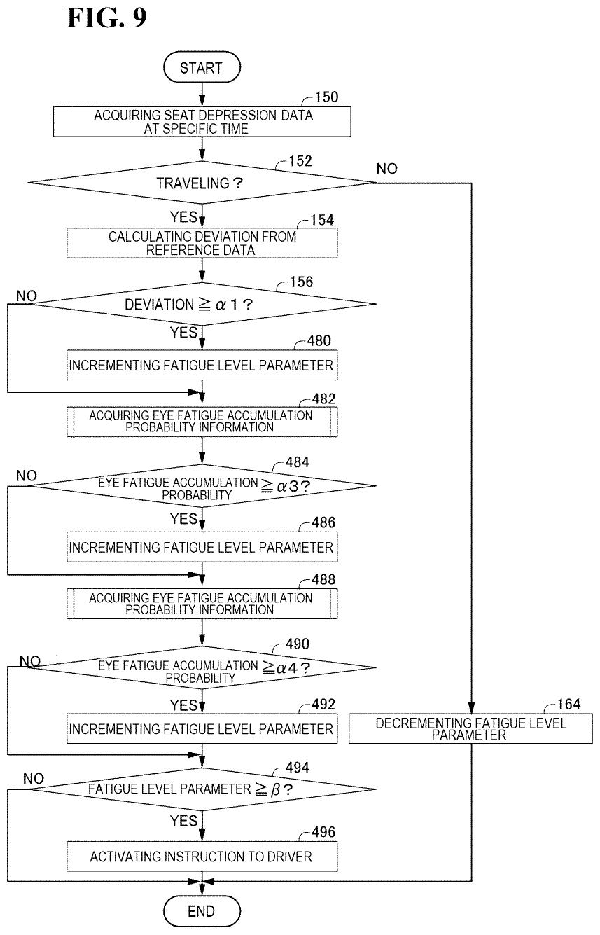

FIG. 9 shows a control structure of a program for implements the fatigue level estimation unit 340 according to the second embodiment. Referring to FIG. 9, this program includes step 150, step 152, step 154 and step 156 similar to those of FIG. 3, and step 480 of incrementing the physical fatigue level parameter when the determination in step 156 is YES.

The program further includes step 482, which is executed when the determination in step 156 is NO and after the determination in step 156 is YES and step 480 is executed, for acquiring the mental fatigue accumulation probability information.

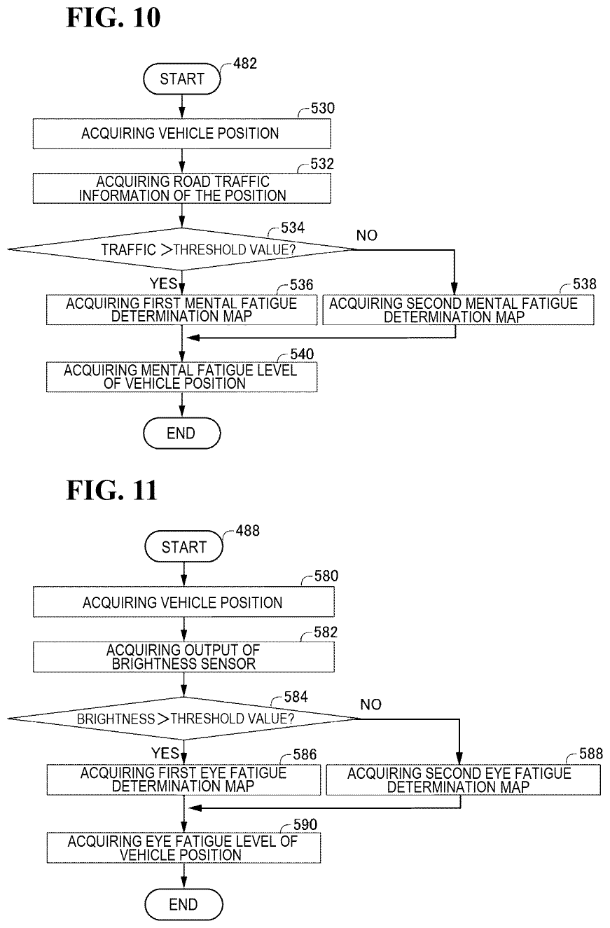

FIG. 10 shows the details of step 482. Referring to FIG. 10, step 482 includes step 530 of acquiring a vehicle position, step 532 of acquiring road traffic information of an area including the position according to the position of the vehicle acquired in step 530, and step 534 of branching a control flow according to whether traffic of the position where the vehicle is present is greater than a threshold value according to the road traffic information acquired in step 532.

The program further includes step 536 of acquiring the second mental fatigue determination map 382 from the mental fatigue determination map storage unit 336 when the determination in step 534 is YES, step 538 of acquiring the first mental fatigue determination map 380 when the determination in step 534 is NO, and step 540 of acquiring information (value) indicating the accumulation probability of the mental fatigue at the position of the vehicle based on the second mental fatigue determination map 382 acquired in step 536 or the first mental fatigue determination map 380 acquired in step 538, and ending the execution of the program.

Referring back once again to FIG. 9, the program includes step 484 for branching the flow of control according to whether the mental fatigue accumulation probability acquired in step 482 is greater than or equal to the threshold value α3, step 486 for incrementing the fatigue level parameter when the determination in step 484 is YES, and step 488 for acquiring the eye fatigue accumulation probability information when the determination in step 484 is NO and after the determination in step 484 is YES and step 486 has been completed.

FIG. 11 shows details of step 488 in FIG. 9. Referring to FIG. 11, step 488 includes step 580 of acquiring a vehicle position, step 582 of acquiring sensor data output from the brightness sensor 312, and step 584 of branching the control flow according to whether the brightness acquired in step 582 is greater than a threshold value.

The program further includes step 586 of acquiring the first eye fatigue determination map 430 from the eye fatigue determination map storage unit 338 when the determination in step 584 is YES, step 588 of acquiring the second eye fatigue determination map 432 when the determination in step 584 is NO, and step 590 of acquiring information (value) indicating the accumulation probability of eye fatigue at the position of the vehicle based on the first eye fatigue determination map 430 acquired in step 586 or the second eye fatigue determination map 432 acquired in step 588, and ending the execution of the program.

Referring back once again to FIG. 9, the program further includes step 490 for branching the flow of control according to whether the eye fatigue accumulation probability is greater than or equal to a threshold value α4 based on the eye fatigue accumulation probability information acquired in step 488, and step 492 for incrementing the fatigue level parameter when the determination in step 490 is YES.

The program further includes, step 494 which branches the control flow based on whether the fatigue level parameter is greater than or equal to the threshold value β after step 492, and step 496 of activating an instruction to prompt the driver to take a rest and ending the execution of the program when the determination in step 494 is YES.

When the determination in step 494 is NO, the execution of the program ends immediately.

The program further includes step 164 which decrements the physical fatigue level parameter and ends execution of the program when the determination in step 152 is NO.

The physical fatigue level parameter after being incremented in step 480, step 486, or step 492 and the physical fatigue level parameter after being decremented in step 164 are stored in the fatigue level storage unit 86 shown in FIG. 6, and are read when step 480, step 486, step 492, or step 164 is executed next. It is noted that, the value to be incremented and the value to be decremented may be equal to each other or may be different from each other, as in the first embodiment. These values and the threshold value α1, the threshold value α3, the threshold value α4, and the threshold value β are design specifications that are determined by a designer through, for example, an experiment at the time of design, depending on the execution interval of the program.

According to this embodiment, the program acquires seat depression data of the driver at a predetermined cycle after the driver starts driving (step 150). When the vehicle is not traveling, the program calculates the center-of-gravity position of the body pressure based on the seat depression data, and calculates the distance from the center-of-gravity position of the reference data (step 154). This program increments the fatigue level parameter in step 160 when the deviation is equal to or larger than the threshold value al, where the distance is the deviation.

Similarly, the fatigue level estimation unit 340 acquires the mental fatigue accumulation probability of the driver at a predetermined cycle (step 482). When the value is equal to or larger than the threshold value α3 (the determination in step 484 is YES), the fatigue level estimation unit 340 increments the fatigue level parameter. The fatigue level estimation unit 340 further acquires the eye fatigue accumulation probability of the driver at a predetermined cycle (step 488). When the value is equal to or larger than the threshold value α4 (the determination in step 490 is YES), the fatigue level estimation unit 340 increments the fatigue level parameter in step 492.

The fatigue level estimation unit 340 finally determines whether the fatigue level parameter is greater than or equal to the threshold value β (step 494), and when the determination is YES, the fatigue level estimation unit 340 activates an instruction to prompt the driver to take a rest (step 496).

During the time when the vehicle is stopped while traveling and while the driver is taking a rest, the determination in step 152 is NO, and the fatigue level parameter is decremented in step 164. As a result, it is possible to give an instruction to take a rest to the driver by an objective index including the mental fatigue and the eye fatigue. When sufficient rest is not taken, the fatigue level parameter will not completely reach zero, and the time until the next rest instruction is given will become shorter. Thus, as long as the driver follows the instruction of the in-vehicle system 300, the driver can drive the vehicle with a sufficient rest, which contributes to traffic safety.

It is noted that, in the second embodiment, the fatigue level is estimated in consideration of the mental fatigue level and the eye fatigue level in addition to the physical fatigue level used in the first embodiment. However, the disclosure is not limited to such embodiments. For example, the physical fatigue level and the mental fatigue level, the physical fatigue level and the eye fatigue level, or the mental fatigue and the eye fatigue level may be combined. Other factors may also be incorporated into the estimation of fatigue.

### 3. Third Embodiment

The in-vehicle system 50 according to the first embodiment and the in-vehicle system 300 according to the second embodiment both perform all management of the fatigue level in the in-vehicle apparatus mounted on the vehicle. However, the disclosure is not limited to such embodiments. For example, the present disclosure can also be implemented with the in-vehicle apparatus and a so-called cloud that can communicate wirelessly with the in-vehicle apparatus. In the following embodiment, a fatigue management server is taken as an example of a server that provides a function similar to that of a cloud, but a service that provides a function similar to that of a fatigue management server according to the following embodiment in a form in which hardware is not specified is also included in this embodiment.

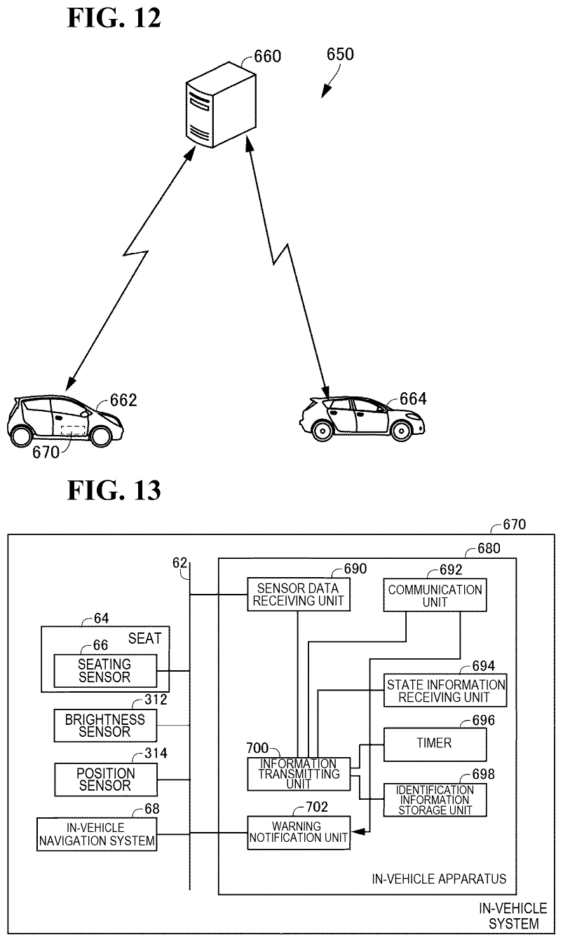

FIG. 12 shows a schematic configuration of a fatigue management system 650 embodies the fatigue level estimation system according to the second embodiment. Referring to FIG. 12, the fatigue management system 650 includes a plurality of vehicles such as a vehicle 662 and a vehicle 664, and a fatigue management server 660. The fatigue management server 660 can communicate with the vehicle 662 and the vehicle 664 via a network including wireless communication, and collects information necessary for estimating the fatigue of the driver of each vehicle from an in-vehicle apparatus mounted on each vehicle (for example, an in-vehicle system 670 of the vehicle 662). The fatigue management server 660 further executes a process necessary for safe driving for the driver who are estimated to have accumulated fatigue.

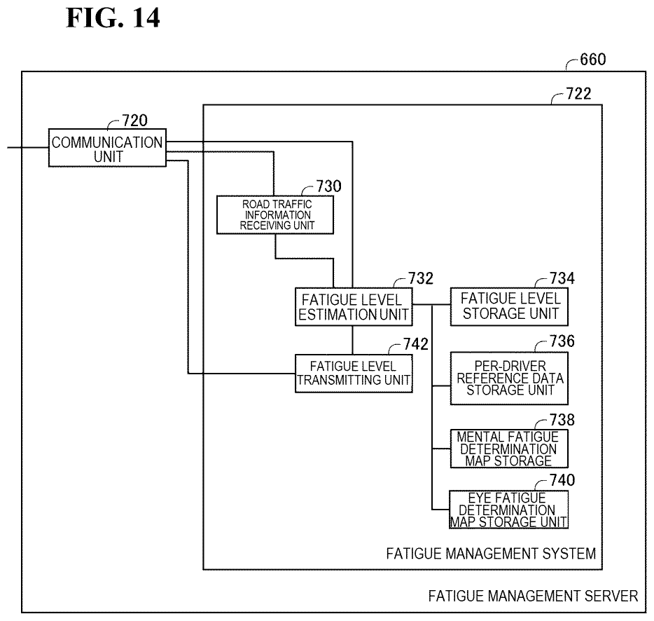

FIG. 13 is a functional block diagram of the in-vehicle system 670 shown in FIG. 12, and FIG. 14 is a functional block diagram of the fatigue management server 660.

Referring to FIG. 13, the in-vehicle system 670 includes the in-vehicle network 62, the seat 64, the seating sensor 66, the in-vehicle navigation system 68, the brightness sensor 312, and the position sensor 314, which are similar to those shown in FIG. 6, as well as an in-vehicle apparatus 680. The in-vehicle apparatus 680 transmits information obtained from the seating sensor 66, the brightness sensor 312, and the position sensor 314 to the fatigue management server 660. The in-vehicle apparatus 680 further executes a process of notifying the driver of a warning to take a rest using the in-vehicle navigation system 68 in response to receiving an instruction to warn the driver to take a rest from the fatigue management server 660.

The in-vehicle apparatus 680 includes a sensor data receiving unit 690, a communication unit 692, a state information receiving unit 694, a timer 696, and an identification information storage unit 698. The sensor data receiving unit 690 is connected to the in-vehicle network 62 and receives sensor data from the seating sensor 66, the brightness sensor 312, and the position sensor 314. The communication unit 692 communicates with the fatigue management server 660. The state information receiving unit 694 receives information indicating the state of the vehicle (traveling, stopped, or parked) from the control unit of the vehicle. The identification information storage unit 698 stores identification information for uniquely identifying a driver of an in-vehicle with the in-vehicle apparatus.

The in-vehicle apparatus 680 further includes an information transmitting unit 700 and a warning notification unit 702. The information transmitting unit 700 periodically executes a process of attaching the state information from the state information receiving unit 694 and the identifier stored in the identification information storage unit 698 to the sensor data collected by the in-vehicle apparatus 680, and transmitting the sensor data to the fatigue management server 660 via the communication unit 692 together with a request for fatigue determination. The information transmitting unit 700 may randomly execute the process. The warning notification unit 702 receives the fatigue level calculated based on the request from the information transmitting unit 700 from the fatigue management server 660 via the communication unit 692. The warning notification unit 702 further issue a warning to prompt the driver to take a rest using the in-vehicle navigation system 68 when the fatigue level is greater than or equal to the threshold value. In this embodiment, when the fatigue level is less than the threshold value, the warning notification unit 702 does nothing in particular. However, the warning notification unit 702 may notify the value of the fatigue level, and may notify the level of the fatigue level by performing display using a color or the like on the screen of the in-vehicle navigation system 68, for example.

Referring to FIG. 14, the fatigue management server 660 includes a communication unit 720 and a fatigue management system 722. The communication unit 720 communicates with each vehicle and other service systems. The fatigue management system 722 receives a fatigue determination request from each vehicle via the communication unit 720. The fatigue management system 722 further executes a process of estimating the fatigue level of the driver of each vehicle and transmitting a reply indicating whether a rest is necessary to the in-vehicle system of each vehicle.

The fatigue management system 722 includes a road traffic information receiving unit 730, a fatigue level storage unit 734, and a per-driver reference data storage unit 736. The road traffic information receiving unit 730 receives road traffic information of each area under the jurisdiction of the fatigue management server 660 from an external road traffic information server via the communication unit 720. The fatigue level storage unit 734 stores a fatigue level for each identifier of each driver. The per-driver reference data storage unit 736 stores reference data for each driver.

The per-driver reference data storage unit 736 stores reference data as shown in FIG. 2 in association with the identifier of each driver. By reading the reference data by designating the identifier of the driver, the reference data for the driver to be subjected to the fatigue determination is obtained from the per-driver reference data storage unit 736.

The fatigue management system 722 further includes a mental fatigue determination map storage unit 738 and an eye fatigue determination map storage unit 740. The maps stored in the mental fatigue determination map storage unit 738 and the eye fatigue determination map storage unit 740 have the same configurations as those shown in FIG. 7 and FIG. 8, respectively. Since the fatigue management server 660 processes a fatigue determination request from a wide range, it is desirable that the maps stored in the mental fatigue determination map storage unit 738 and the eye fatigue determination map storage unit 740 sufficiently cover at least the regions under their jurisdiction. It is noted that, in this embodiment, these are called “map”. However, in practice, any method may be used as long as the probability of the mental fatigue accumulation and the probability of the eye fatigue accumulation corresponding to the position of the vehicle can be obtained from the position of the vehicle. For example, these maps may be databases that include numerous records including of a combination of values or sets of values that define positions within a certain range, along with values indicating the probability of mental fatigue accumulation and values indicating the probability of eye fatigue accumulation.

The fatigue management system 722 further includes a fatigue level estimation unit 732 and a fatigue level transmitting unit 742. The fatigue level estimation unit 732 receives a fatigue determination request from the vehicle via the communication unit 720. The fatigue level estimation unit 732 further estimates the fatigue level of the driver by using the sensor data and the driver identifier included in the fatigue determination request, the road traffic information from the road traffic information receiving unit 730, and the reference data stored in association with the driver identifier included in the received fatigue determination request, which is read from the per-driver reference data storage unit 736, and stores the fatigue level in the fatigue level storage unit 734. The fatigue level transmitting unit 742 transmits the estimated fatigue level to the in-vehicle system that is the transmission source of the fatigue determination request.

This embodiment is different from the first embodiment or the second embodiment in that all the estimation of the substantial fatigue level is performed in the fatigue management server 660, and the in-vehicle system 670 simply collects sensor data and provides a function of requesting the fatigue management server 660 to perform the fatigue estimation. However, according to the third embodiment, it is possible to prompt the driver to take a rest according to an objective criterion, which contributes to traffic safety.

### 4. Implementation by Computer

The in-vehicle apparatus according to the first embodiment, the second embodiment, and the third embodiment can be achieved through computer hardware and the software executed by that hardware.

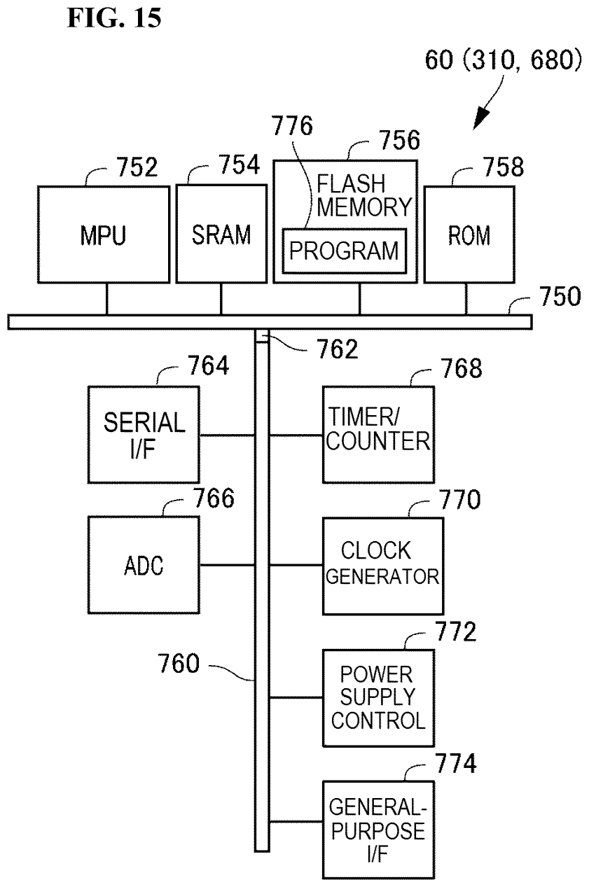

FIG. 15 shows a configuration of the in-vehicle apparatus 60 according to the first embodiment, for example. It is noted that, the in-vehicle apparatus 310 and the in-vehicle apparatus 680 can also be achieved with the same configuration.

Referring to FIG. 15, the in-vehicle apparatus 60 is substantially a computer, and includes a micro processing unit (MPU) 752 as a processor, a high-speed bus 750 to which the MPU 752 is connected, a static random access memory (SRAM) 754 connected to high-speed bus 750, a flash memory 756 connected to high-speed bus 750, and a read-only memory (ROM) 758 connected to high-speed bus 750. The SRAM 754 holds data necessary for execution of the program. The flash memory 756 stores a program 776 for implementing the functions implemented by the in-vehicle apparatus 60, the in-vehicle apparatus 310, and the in-vehicle apparatus 680. The ROM 758 stores a boot-up program of the MPU 752 and the like.

The MPU further includes a low-speed bus 760 connected to the high-speed bus 750 via a bridge 762, and a serial interface (I/F) 764, an ADC (analog-to-digital converter) 766, a timer/counter 768, a clock generator 770, a power supply control unit 772, and a general-purpose I/F 774, all of which are connected to the low-speed bus 760.

It is noted that, since the operation of the MPU is well known and the function of the program executed by the MPU is significant in the embodiment, the operation of the MPU itself will not be described in the following description.

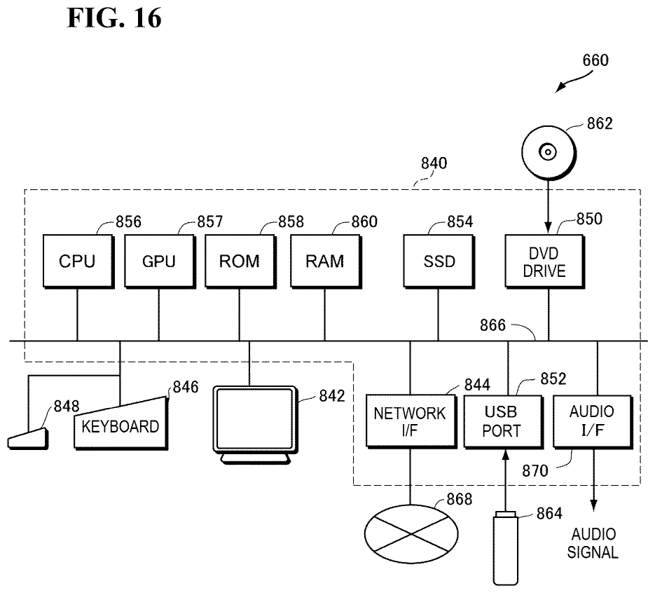

FIG. 16 is a hardware block diagram of a computer system that operates as the fatigue management server 660 shown in FIG. 14. Referring to FIG. 16, the fatigue management server 660 includes a computer 840 having a DVD (digital versatile disc) drive 850, and a keyboard 846, a mouse 848, and a monitor 842, all of which are connected to the computer 840, for interaction with the user. Of course, these are merely examples of configurations for when user interaction is required, and any general hardware and software that can be used for user interaction (for example, touch panels, voice inputs, pointing devices in general) can be used.

The computer 840 includes a CPU (central processing unit) 856, a GPU (graphics processing unit) 857, and a bus 866 connected to the CPU 856, the GPU 857, and the DVD drive 850, in addition to the DVD drive 850. The computer 840 further includes a ROM 858 connected to the bus 866 and storing a boot-up program of the computer 840, a RAM (random access memory) 860 connected to the bus 866 and storing instructions including a program, a system program, work data, and the like, and an SSD (solid state drive) 854 which is a nonvolatile memory connected to the bus 866. The SSD 854 is for storing programs executed by the CPU 856 and the GPU 857, data used by the programs executed by the CPU 856 and the GPU 857, and the like. The computer 840 further includes a network I/F 844 that provides a connection to a network 868 that enables communication with other terminals, an USB port 852 to which an USB (universal serial bus) memory 864 is attachable and from which the USB memory 864 and each unit in the computer 840 communicate with each other, and an audio I/F 870 connected to the bus 866.

In the above embodiment, programs for implementing the functions of the fatigue management server 660 shown in FIG. 14 are stored in, for example, the SSD 854, the RAM 860, a DVD 862, or the USB memory 864 shown in FIG. 16, or a storage medium of an external device (not shown) connected via the network I/F 844 and the network 868. Typically, these data, parameters, and the like are written to the SSD 854 from the outside, for example, and are loaded into the RAM 860 when the computer 840 is executed.

The computer program designed to operate this computer system to enable the functions of the fatigue management server 660 and its components, as shown in FIG. 14, is stored on the DVD 862 that is mounted in the DVD drive 850 and transferred from the DVD drive 850 to the SSD 854. Alternatively, these programs are stored in the USB memory 864, the USB memory 864 is attached to the USB port 852, and the programs are transferred to the SSD 854. Alternatively, the program may be transmitted to the computer 840 through the network 868 and stored in the SSD 854.

The program is loaded into the RAM 860 at the time of execution. Of course, the keyboard 846, the monitor 842, and the mouse 848 may be used to input the source program, and the object program after compilation may be stored in the SSD 854.

The CPU 856 reads a program from the RAM 860 according to an address indicated by a register (not shown) called a program counter inside the CPU 856, interprets a command, reads data necessary for execution of the command from the RAM 860, the SSD 854, or another device according to an address designated by the command, and executes a process designated by the command. The CPU 856 stores the data of the execution result in an address designated by the program, such as the RAM 860, the SSD 854, or a register in the CPU 856. Depending on the address, the command is output from the computer as a command to an actuator of the robot, a voice signal, or the like. At this time, the value of the program counter is also updated by the program. The computer program may be loaded directly into the RAM 860 from the DVD 862, from the USB memory 864, or via the network 868. It is noted that, in the program executed by the CPU 856, some tasks (mainly numerical calculations) are dispatched to the GPU 857 by instructions included in the program or according to analysis results at the time of instruction execution by the CPU 856.

The program for the fatigue management server 660 and its respective units according to the above-described embodiment, which is executed by the computer 840, includes a plurality of instructions that are written and arranged to operate the computer 840 in a way that enables those functions. Some of the basic functions required to execute the instructions may be provided by an operating system (OS) or a third party program running on the computer 840, or by an execution environment of various toolkit modules or programs installed on the computer 840. Thus, the program does not necessarily have to include all the functions required to implement the system and method of this embodiment. The program may include only instructions for executing the operations of the above-described devices and components thereof by statically linking appropriate functions or modules during compilation or dynamically calling them during execution in a manner controlled to obtain a desired result. The operation method of the computer 840 for this purpose is well known, and thus, will not be repeated here.

It is noted that, the GPU 857 can perform parallel processes, and can execute a large amount of calculations accompanying the fatigue level estimation requests from a plurality of oblique-viewing apparatuses simultaneously in parallel or in a pipeline manner. For example, parallel computation elements found in a program at the time of compiling the program or parallel computation elements found at the time of executing the program are dispatched from the CPU 856 to the GPU 857 and executed as needed, and the results are returned to the CPU 856 directly or via a predetermined address of the RAM 860 and substituted into a predetermined variable in the program.

The process (each function) of each of the above-described embodiments are implemented by a process circuitry that includes one or more processors. The process circuitry may be configured by an integrated circuit or the like in which one or more memories, various analog circuitries, and various digital circuitries are combined in addition to the one or more processors. The one or more memories store a program (instruction) that causes the one or more processors to execute each of the processes. The one or more processors may execute the processes in accordance with the program read from the one or more memories, or may execute the processes in accordance with a logic circuitry designed in advance to execute the processes. The processor may be various processors suitable for control of a computer, such as a CPU, a GPU, a digital signal processor (DSP), a field-programmable gate array (FPGA), and an application specific integrated circuit (ASIC). The plurality of physically separated processors may execute the processes in cooperation with each other. For example, the processors each mounted on a plurality of computers that are physically separated from each other may execute the processes in cooperation with each other via a network such as a local area network (LAN), a wide area network (WAN), or the Internet. The program may be installed in the memory from an external server device or the like via the network, or may be distributed in a state of being stored in a recording medium such as a compact disc read-only memory (CD-ROM), a digital versatile disc read-only memory (DVD-ROM), or a semiconductor memory, and installed in the memory from the recording medium.

The embodiments disclosed herein are to be considered in all respects as illustrative and not restrictive. The scope of the present disclosure is indicated by the appended claims rather than by the foregoing description, and all changes that come within the meaning and range of equivalency of the claims are intended to be embraced therein.

### REFERENCE SIGNS LIST

50, 300, 670 in-vehicle system
60, 310, 680 in-vehicle apparatus
62 in-vehicle network
64 seat
66 seating sensor
68 in-vehicle navigation system
80, 330, 690 sensor data receiving unit
82, 694 state information receiving unit
84, 696 timer
86, 734 fatigue level storage unit
88 reference data storage unit
90, 340, 732 fatigue level estimation unit (calculation unit, addition unit, measuring unit, determination unit)
92, 342, 702 warning notification unit
120 reference data
130, 210 center of gravity
200 seat depression data
212 distance
250, 252, 260, 262 region
270, 272 difference
312 brightness sensor
314 position sensor
332, 692, 720 communication unit
334, 730 road traffic information receiving unit
336, 738 mental fatigue determination map storage unit
338, 740 eye fatigue determination map storage unit
380 first mental fatigue determination map
382 second mental fatigue determination map
390 road
392, 434 determination
430 first eye fatigue determination map
432 second eye fatigue determination map
650, 722 fatigue management system
660 fatigue management server
662, 664 vehicle
698 identification information storage unit
700 information transmitting unit
736 per-driver reference data storage unit
742 fatigue level transmitting unit
750 high-speed bus
752 MPU
754 SRAM
756 flash memory
758, 858 ROM
760 low-speed bus
762 bridge
764 serial I/F
766 ADC
768 timer/counter
770 clock generator
772 power supply control unit
774 general-purpose I/F
776 program
840 computer
842 monitor
844 network I/F
846 keyboard
848 mouse
850 DVD drive
852 USB port
854 SSD
856 CPU
857 GPU
860 RAM
862 DVD
864 USB memory
866 bus
868 network
870 audio I/F

1. An in-vehicle apparatus to be mounted on a vehicle including a seat and a seating sensor provided on the seat, the in-vehicle apparatus comprising:
a seating sensor output receiving circuitry configured to receive sensor data from the seating sensor;
a state information receiving circuitry configured to receive state information indicating a state of the vehicle; and
a fatigue level estimation circuitry connected to the seating sensor output receiving circuitry and the state information receiving circuitry and configured to estimate a fatigue level of a driver of the vehicle seated on the seat provided with the seating sensor, based on the sensor data and the state information.

2. The in-vehicle apparatus according to claim 1, wherein
the fatigue level estimation circuitry includes:
a fatigue level increasing circuitry configured to increase a fatigue level parameter based on the sensor data when the state information indicates that the vehicle is traveling; and
a fatigue level decreasing circuitry configured to decrease the fatigue level parameter when the state information indicates that the vehicle is not traveling.

3. The in-vehicle apparatus according to claim 2, wherein
the fatigue level increasing circuitry includes:
a deviation calculation circuitry configured to repeatedly calculate a deviation determined between the sensor data and reference data over a period of time during the state information indicating that the vehicle is traveling, the reference data being acquired in advance from a sitting posture leading to less fatigue;
a first determination circuitry configured to determine whether the deviation is greater than or equal to a threshold value in response to the deviation being calculated by the deviation calculation circuitry; and
an addition circuitry configured to add a first value to the fatigue level in response to a determination by the first determination circuitry that the deviation is greater than or equal to the threshold value.

4. The in-vehicle apparatus according to claim 3, wherein
the reference data includes center-of-gravity position information indicating a first center-of-gravity position of a seated person in the sitting posture leading to less fatigue, the first center-of-gravity position being based on body pressure exerted on the seating sensor by the seated person, and
the deviation calculation circuitry includes:
a deviation storage circuitry configured to store the deviation;
a center-of-gravity position calculation circuitry configured to calculate a second center-of-gravity position of the driver based on body pressure exerted on the seating sensor by the driver, based on the sensor data over the period of time during the state information indicating that the vehicle is traveling;
a distance calculation circuitry configured to calculate a distance defined between the first center-of-gravity position and the second center-of-gravity position; and
a distance addition circuitry configured to add the distance calculated by the distance calculation circuitry to the deviation stored in the deviation storage circuitry.

5. The in-vehicle apparatus according to claim 3, wherein
the reference data includes a reference distribution, the reference distribution being a distribution of body pressure exerted on the seating sensor by a seated person in the sitting posture leading to less fatigue, and
the deviation calculation circuitry includes:
a deviation storage circuitry configured to store the deviation;
a body pressure distribution measuring circuitry configured to measure a body pressure distribution of the driver, based on the sensor data over the period of time during the state information indicating that the vehicle is traveling, the body pressure distribution being a distribution of body pressure exerted on the seating sensor by the driver;
a distance calculation circuitry configured to calculate a distance defined between the body pressure distribution and the reference distribution; and
a distance addition circuitry configured to add the distance calculated by the distance calculation circuitry to the deviation stored in the deviation storage circuitry.

6. The in-vehicle apparatus according to claim 2, wherein
the fatigue level increasing circuitry includes:
a fatigue level storage circuitry configured to store a value of the fatigue level; and
a movement amount calculation circuitry configured to calculate an amount of movement of a center-of-gravity position of the driver based on body pressure exerted on the seating sensor by the driver, based on the sensor data over a period of time during the state information indicating that the vehicle is traveling, and add the amount of movement to the value of the fatigue level.

7. The in-vehicle apparatus according to claim 3, wherein
the fatigue level estimation circuitry further includes:
a first probability calculation circuitry configured to calculate a probability of mental fatigue accumulation in the driver, based on a traveling environment of the vehicle;
a second determination circuitry configured to determine whether the probability calculated by the first probability calculation circuitry is greater than or equal to a threshold value; and
a mental fatigue level addition circuitry configured to add a second value to the fatigue level in response to a determination by the second determination circuitry that the probability is greater than or equal to the threshold value.

8. The in-vehicle apparatus according to claim 7, wherein
the fatigue level estimation circuitry further includes:
a second probability calculation circuitry configured to calculate a probability of eye fatigue accumulation in the driver, based on the traveling environment of the vehicle;
a third determination circuitry configured to determine whether the probability calculated by the second probability calculation circuitry is greater than or equal to a threshold value; and
an eye fatigue level addition circuitry configured to add a third value to the fatigue level in response to a determination by the third determination circuitry that the probability is greater than or equal to the threshold value.

9. The in-vehicle apparatus according to claim 3, wherein
the fatigue level estimation circuitry further includes:
a first probability calculation circuitry configured to calculate a probability of eye fatigue accumulation in the driver, based on a traveling environment of the vehicle;
a second determination circuitry configured to determine whether the probability calculated by the first probability calculation circuitry is greater than or equal to a threshold value; and
an eye fatigue level addition circuitry configured to add a second value to the fatigue level in response to a determination by the second determination circuitry that the probability is greater than or equal to the threshold value.

10. A fatigue level estimation system for estimating a fatigue level of a driver of a vehicle including a seating sensor, the fatigue level estimation system comprising an in-vehicle apparatus to be mounted on the vehicle, and a server configured to communicate with the in-vehicle apparatus,
the in-vehicle apparatus including:
a seating sensor output receiving circuitry configured to receive sensor data from the seating sensor;
a state information receiving circuitry configured to receive state information indicating a state of the in-vehicle with the in-vehicle apparatus; and
a transmitting circuitry configured to transmit the sensor data received by the seating sensor output receiving circuitry and the state information received by the state information receiving circuitry to the server,
the server including:
a fatigue level estimation circuitry configured to receive the sensor data and the state information from the in-vehicle apparatus and estimate a fatigue level of the driver of the vehicle seated on a seat provided with the seating sensor, based on the sensor data and the state information; and
a process execution circuitry configured to execute a predetermined process for reducing fatigue of the driver in response to the fatigue level estimated by the fatigue level estimation circuitry exceeding a threshold value.

11. A fatigue level estimation method for estimating a fatigue level of a driver seated on a seat of a vehicle based on sensor data from a seating sensor provided on the seat, the fatigue level estimation method comprising:
receiving the sensor data from the seating sensor;
receiving state information indicating a state of the vehicle; and
estimating a fatigue level of the driver based on the sensor data and the state information.

12. A non-transitory storage medium storing computer program for causing a computer to estimate a fatigue level of a driver seated on a seat of a vehicle based on sensor data from a seating sensor provided on the seat,
the computer program causing the computer to execute:
receiving the sensor data from the seating sensor;
receiving state information indicating a state of the vehicle; and
estimating a fatigue level of the driver based on the sensor data and the state information.

13. The in-vehicle apparatus according to claim 3, wherein
the fatigue level increasing circuitry includes:
a fatigue level storage circuitry configured to store a value of the fatigue level; and
a movement amount calculation circuitry configured to calculate an amount of movement of a center-of-gravity position of the driver based on body pressure exerted on the seating sensor by the driver, based on the sensor data over a period of time during the state information indicating that the vehicle is traveling, and add the amount of movement to the value of the fatigue level.

14. The in-vehicle apparatus according to claim 4, wherein
the fatigue level increasing circuitry includes:
a fatigue level storage circuitry configured to store a value of the fatigue level; and
a movement amount calculation circuitry configured to calculate an amount of movement of a center-of-gravity position of the driver based on body pressure exerted on the seating sensor by the driver, based on the sensor data over a period of time during the state information indicating that the vehicle is traveling, and add the amount of movement to the value of the fatigue level.

15. The in-vehicle apparatus according to claim 5, wherein
the fatigue level increasing circuitry includes:
a fatigue level storage circuitry configured to store a value of the fatigue level; and
a movement amount calculation circuitry configured to calculate an amount of movement of a center-of-gravity position of the driver based on body pressure exerted on the seating sensor by the driver, based on the sensor data over a period of time during the state information indicating that the vehicle is traveling, and add the amount of movement to the value of the fatigue level.

16. The in-vehicle apparatus according to claim 4, wherein
the fatigue level estimation circuitry further includes:
a first probability calculation circuitry configured to calculate a probability of mental fatigue accumulation in the driver, based on a traveling environment of the vehicle;
a second determination circuitry configured to determine whether the probability calculated by the first probability calculation circuitry is greater than or equal to a threshold value; and
a mental fatigue level addition circuitry configured to add a second value to the fatigue level in response to a determination by the second determination circuitry that the probability is greater than or equal to the threshold value.

17. The in-vehicle apparatus according to claim 5, wherein
the fatigue level estimation circuitry further includes:
a first probability calculation circuitry configured to calculate a probability of mental fatigue accumulation in the driver, based on a traveling environment of the vehicle;
a second determination circuitry configured to determine whether the probability calculated by the first probability calculation circuitry is greater than or equal to a threshold value; and
a mental fatigue level addition circuitry configured to add a second value to the fatigue level in response to a determination by the second determination circuitry that the probability is greater than or equal to the threshold value.

18. The in-vehicle apparatus according to claim 6, wherein
the fatigue level estimation circuitry further includes:
a first probability calculation circuitry configured to calculate a probability of mental fatigue accumulation in the driver, based on a traveling environment of the vehicle;
a second determination circuitry configured to determine whether the probability calculated by the first probability calculation circuitry is greater than or equal to a threshold value; and
a mental fatigue level addition circuitry configured to add a second value to the fatigue level in response to a determination by the second determination circuitry that the probability is greater than or equal to the threshold value.

19. The in-vehicle apparatus according to claim 4, wherein
the fatigue level estimation circuitry further includes:
a first probability calculation circuitry configured to calculate a probability of eye fatigue accumulation in the driver, based on a traveling environment of the vehicle;
a second determination circuitry configured to determine whether the probability calculated by the first probability calculation circuitry is greater than or equal to a threshold value; and
an eye fatigue level addition circuitry configured to add a second value to the fatigue level in response to a determination by the second determination circuitry that the probability is greater than or equal to the threshold value.

20. The in-vehicle apparatus according to claim 5, wherein
the fatigue level estimation circuitry further includes:
a first probability calculation circuitry configured to calculate a probability of eye fatigue accumulation in the driver, based on a traveling environment of the vehicle;
a second determination circuitry configured to determine whether the probability calculated by the first probability calculation circuitry is greater than or equal to a threshold value; and
an eye fatigue level addition circuitry configured to add a second value to the fatigue level in response to a determination by the second determination circuitry that the probability is greater than or equal to the threshold value.
